(19) United States
(12) Patent Application Publication
(10) Pub. No.: US 2024/0417425 A1
(43) Pub. Date: Dec. 19, 2024

(54) CARDIAC STEROID DERIVATIVES

(71) Applicant: YISSUM RESEARCH DEVELOPMENT COMPANY OF THE HEBREW UNIVERSITY OF JERUSALEM LTD., Jerusalem (IL)

(72) Inventors: David LICHTSTEIN, Shoeva (IL); Joseph DEUTSCH, Jerusalem (IL)

(21) Appl. No.: 18/718,915

(22) PCT Filed: Dec. 12, 2022
(86) PCT No.: PCT/IL2022/051306

§ 371 Date: Jun. 12, 2024

Related U.S. Application Data
(60) Provisional application No. 63/265,588, filed on Dec. 17, 2021.

(51) Int. Cl. C07J 19/00; A61K 31/585; A61P 31/14; A61P 35/00

(52) U.S. Cl. CPC C07J 19/00; A61K 31/585; A61P 31/14; A61P 35/00

(57) ABSTRACT

The technology disclosed herein concerns de-hydroxylated cardiac steroid of formula (I) and uses thereof in medicine.

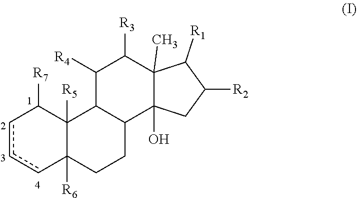

### TECHNOLOGICAL FIELD

The invention generally concerns new cardiac steroids, methods for their preparation and uses thereof.

### BACKGROUND

Cardiac steroids are commonly used for the treatment of congestive heart failure and atrial fibrillation. They inhibit Na⁺, K⁺-ATPase activity, thus resulting in increased intracellular sodium and decreased intracellular potassium. In turn, the increased levels of sodium blocks the antiporter activity of the Na⁺/Ca²⁺ exchanger, resulting in accumulation of Ca²⁺ within the endoplasmic reticulum and mitochondria. Eventually, this allows for an increased release of Ca²⁺ from the endoplasmic reticulum in response to contraction stimuli, which in turn increase the output force of the heart and increase its rate of contractions. The excessive intracellular calcium can result in delayed after-depolarizations, which may result in dysrhythmias.

Toxicity of cardiac steroids can be acute or chronic. Acute toxicity often occurs following an acute overdose and is more likely to result in younger individuals. The most common symptoms of such toxicity include dysrhythmias, nausea, vomiting, hyperkalemia, abdominal pain and visual changes. Toxicity of chronic cardiac steroids, in contrast to acute toxicity, frequently occurs in the elderly as a result of a decreased renal clearance which can be a result of a drug-drug interaction or age-related renal function decline. The most common symptoms of chronic cardiac steroids toxicity are weakness, nausea and malaise. The gastrointestinal symptoms are less common than in acute toxicity, but neurological manifestations, such as fatigue, confusion, lethargy and weakness are more common.

### SUMMARY OF THE INVENTION

To overcome toxicities associated with the use of cardiac steroids, the inventors of the technology disclosed herein have developed a new class of steroids which are less toxic and more effective as compared to their existing analogs.

As known in the art, cardiac steroids such as digoxin, ouabain and bufalin are highly toxic at therapeutic doses.

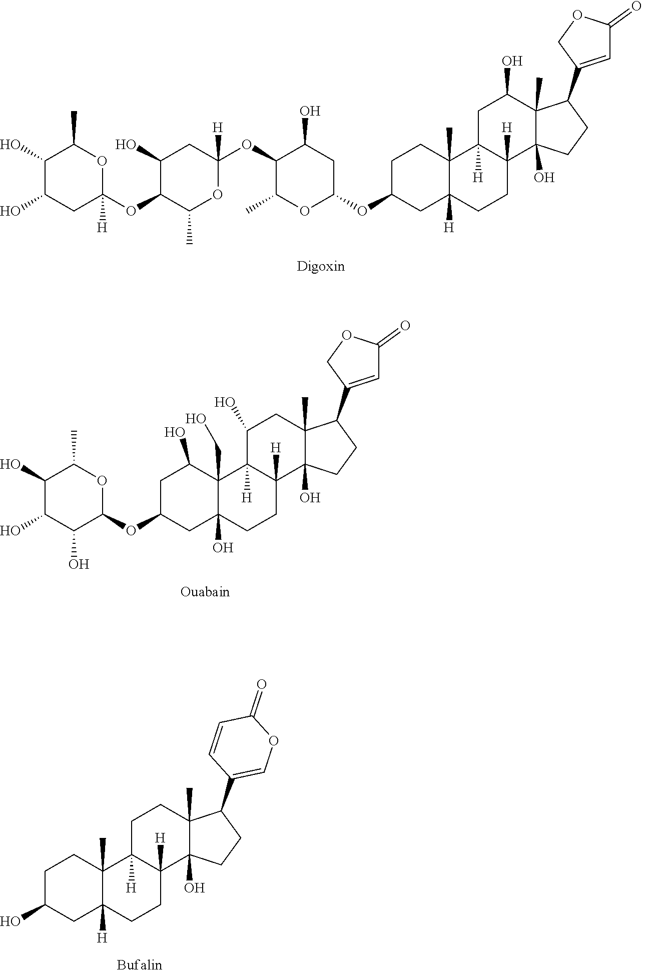

Cardiac Steroids are composed of three major structural components: a steroid core, in which rings AB and CD are cis-fused, whereas rings BC are trans-fused: a 5- and 6-membered lactone ring at position 17 (indicated by variant R, reflecting cardenolides and bufadienolides, respectively); and a variable number of sugar residues are bonded through an oxygen atom at position 3. Thus, common to all cardiac steroids having the general structure below is the presence of an oxygen-bearing group at position 3 (the C3 position of ring A). A plurality of various substituents selected from —H, —OR′, —C₁-C₅alkyl, —C₁-C₅alkylene-hydroxyl, —C(═O)R″, and others, as defined herein, may be provided on any of the caron atoms of the ring structure of the cardiac steroids. This plurality, i.e., one or more of substituents, is designated by a variant X in the structure below. The selection of substituents is exemplified in a compound of structure (I) below.

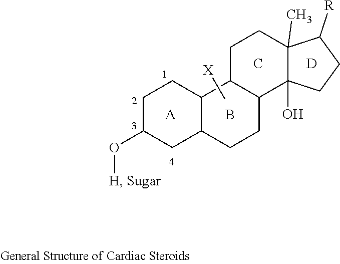

The inventors of the present invention have realized that elimination of the oxygen atom from the C3 position, and by that eliminating any substitution from the C3 position, results in a novel class of compounds having high affinities to Na+, K− ATPase, rendering the compound potent cardiotonic steroids.

Thus, in a first aspect the invention provides a C3 de-hydroxy cardiac steroid. In other words, in a general structure shown above for cardiac steroids, the C3 oxygen bearing group, e.g., —OH group in the case of bufalin and similar compounds and —O—sugar in the case of digoxin and ouabain, is absent. In compounds of the invention, the SP³ hybridization of the carbon at position C3 is transformed into an SP² hybridization, forming an endo-cyclic double bond between either C2 and C3 or between C3 and C4 of ring A. In most general terms, compounds of the invention may be described by the compound of structure

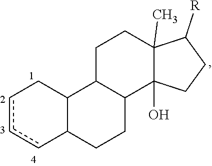

as further defined with respect to a compound of structure (I).

Thus, the C3-dehydroxy cardiac steroid is a compound having a structure

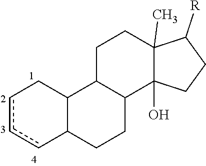

further substituted as shown below.

In some embodiments, the C3-dehydroxy cardiac steroid is of structure (I) as defined below.

Compounds of the invention are cardiotonic steroids.

As explained herein, a cardiac steroid is an organic compound that is medicinally useful in increasing rate of contractions and output force of the heart by interacting with Na⁺, K⁺-ATPase. Typical steroids present a structural backbone such as that shown above, wherein position 3 (C3 position) of the steroid backbone is typically substituted with a hydroxyl group (—OH) or with a derivative thereof, e.g., an ether group, wherein the oxygen atom bonds to a sugar moiety. Unlike these typical steroids, compounds of the invention lack the hydroxyl group at position 3 or are free of a C3 O-sugar group and are therefore regarded as being de-hydroxylated or regarded as de-hydroxy cardiac steroids.

The cardiac steroid of the invention may be described by a compound having the structure of general formula (I):

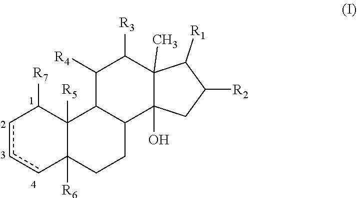

wherein
R₁ is a lactone,
each of R₂, R₃, R₄, R₅, R₆ and R₇, independently of the other, may be selected from —H, —OR′, —C₁-C₅alkyl, —C₁-C₅alkylene-hydroxyl and —C(═O)R″, each of R′ and R″, independently of the other, may be selected from —H, —C₁-C₅alkyl, —C₁-C₅alkylene-hydroxyl and —C(═O)C₁-C₅alkyl; and
wherein one of the bonds designated [symbol] (namely the bond between C2 and C3 and the bond between C3 and C4) is a double bond and the other is a single bond (in other words, each of a bond between C2 and C3 and a bond between C3 and C4 designated [symbol] is a double bond or a single bond, such that when the bond between C3 and C4 is a double bond, the bond between C3 and C2 and a single bond, and vice versa).

As used herein, R₁ is selected amongst lactone functionalities; namely amongst ring structures which contain an ester group. In some embodiments, the lactone may be a 5-memebred ring or a 6-memebered ring, namely comprising 5 or 6 ring atoms, respectively, one of which being an oxygen atom (the lactone oxygen). The lactone ring may optionally comprise one additional heteroatom selected from N, O and S.

In some embodiments, the lactone ring, independent of its size and presence of one or more additional heteroatoms, comprises one or two endocyclic double bonds.

In some embodiments, the lactone ring, being variant R1, may be lactone (i) or lactone (ii):

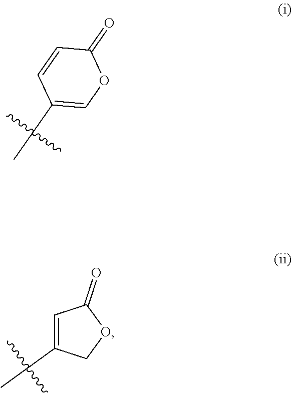

wherein [symbol] is a point (a bond) of connectivity to the backbone structure of compound (I).

In some embodiments, a compound of the general formula (I), is a compound of formula (Ia) or formula (Ib):

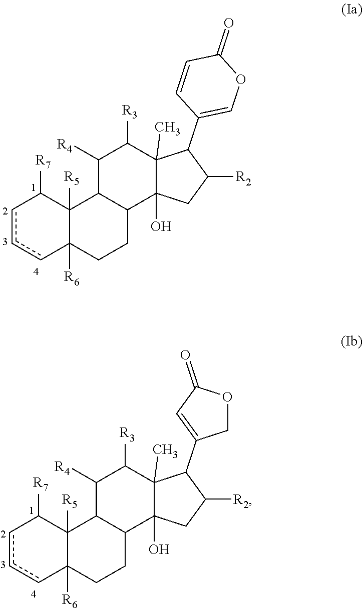

wherein each of R₂, R₃, R₄, R₅, R₆, R₇, R′, R″ and [symbol] is as defined herein.

Each of R₂, R₃, R₄, R₅, R₆ and R₇, independently of the other, may be selected from —H, —OR′, —C₁-C₅alkyl, —C₁-C₅alkylene-hydroxyl and —C(═O)R″, and each of R′ and R″, independently of the other, may be selected from —H, —C₁-C₅alkyl, —C₁-C₅alkylene-hydroxyl and —C(—O)C₁-C₅alkyl.

Variant-OR′ represents a hydroxyl group (—OH) when R′ is-H, or an ether group when R′ is different from —H. Wherein R′ is —C₁-C₅alkyl, —C₁-C₅alkylene-hydroxyl or —C(—O)C₁-C₅alkyl, —OR′ may be selected amongst —O—C₁-C₅alkyl, —O—C₁-C₅alkylene-hydroxyl and —O—C(═O)C₁-C₅alkyl.

The group “—C₁-C₅alkyl” represents an alkyl group having between 1 and 5 carbon atoms, inclusive. The alkyl may be linear or substituted. Non-limiting examples of the alkyl group include methyl, ethyl, propyl, butyl and pentyl. The group “—C₁-C₅alkylene-hydroxyl” similarly represents an alkylene group having between 1 and 5 carbon atoms, wherein one or more of the carbon atoms of the alkylene is/are substituted with one or more hydroxyl groups. Non-limiting examples include —CH₃—OH, —CH₂CH(OH)CH₃, —CH(OH)CH₂CH₃, and —CH₂CH₂CH₂OH.

In some embodiments, the —C₁-C₅alkylene-hydroxyl also encompasses diols of the form —CH(OH)₂, —CH(OH)CH(OH)CH₃, —CH(OH)CH₂CH₂OH, —CH₂CH(OH)CH₂OH and —CH₂CH(OH)CH₂OH.

The group-C(═O) R″ represents an aldehyde when R″ is-H or a ketone group when R″ is an alkyl group. Wherein R″ is —C₁-C₅alkyl, —C₁-C₅alkylene-hydroxyl or —C(═O)C₁-C₅alkyl, the group —C(═O)R″ is —C(═O)—C₁-C₅alkyl or —C(═O)—C₁-C₅alkylene-hydroxyl.

In a compound of formula (I), the bond designated [symbol] may be a double bond or a single bond. When the bond between C3 and C4 is a double bond, the bond between C3 and C2 is a single bond, and when the bond between C3 and C4 is a single bond, the bond between C3 and C2 is a double bond.

In some embodiments, in a compound of general formula (Ia) or (Ib), R₂ is selected from H and OR′.

In some embodiments, R′ is —H. In some embodiments, R′ is —C(═O)C₁-C₅alkyl.

In some embodiments, the —C(═O)C₁-C₅alkyl is —C(═O)CH₃.

In some embodiments, in a compound of general formula (Ia) or (Ib), R₃ is selected from —H and —OH. In some embodiments, R₃ is —H.

In some embodiments, in a compound of general formula (Ia) or (Ib), R₄ is selected from —H and —OH. In some embodiments, R₄ is —H.

In some embodiments, in a compound of general formula (Ia) or (Ib), R₅ is selected from —H, —C₁-C₅alkyl, —C₁-C₅alkylene-hydroxyl and —C(═O) R′, wherein R′ is selected from —H and —C₁-C₅alkyl. In some embodiments, R₅ is —C₁-C₅alkyl, which is optionally-CH₃.

In some embodiments, in a compound of general formula (Ia) or (Ib), R₆ is selected from —H and —OH. In some embodiments, R₆ is —H.

In some embodiments, in a compound of general formula (Ia) or (Ib), R₇ is selected from —H and —OR′, wherein R′ is selected from —H and C₁-C₅alkyl. In some embodiments, R₇ is —H.

In some embodiments, in a compound of general formula (Ia) or (Ib), the bond between C3 and C2 is a double bond or the bond between C3 and C4 is a double bond.

In some embodiments, in a compound of formula (I), (Ia) or (Ib) the double bond is between C3 and C2 or between C3 and C4.

In some embodiments, in a compound of formula (Ia), each of R₂, R₃, R₄, R₆ and R₇ is-Hand R₅ is-CH₃.

In another aspect, the invention provides a compound having the structure:

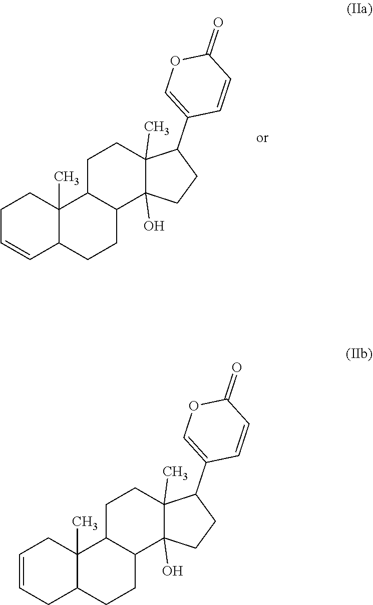 [SMILES: CC12CCC=CC1CCC1C2CCC2(C)C(c3ccc(=O)oc3)CCC12O ; CC12CC=CCC1CCC1C2CCC2(C)C(c3ccc(=O)oc3)CCC12O]

The invention further contemplates use of a compound according to the invention in medicine.

The plasma membrane Na⁺ and K⁺ transporter Na⁺, K⁺-ATPase is an established receptor for cardiac steroids. The interaction of these steroids with Na⁺, K⁺-ATPase results in inhibition of the ion-pumping function and, in addition, causes the activation of several signal transduction cascades, including mitogen-activated protein kinase: extracellular signal-regulated kinase: proto-oncogene tyrosine-protein kinase (Src): PI3K/Akt, Ca⁺² signaling, and reactive oxygen species generation pathways. It is well established that the toxicity of cardiac steroids in the heart is due to calcium overload, produced by excessive inhibition of the Nat, K⁺-ATPase in the myocytes, leading to arrhythmia and lethality. Conversely, the positive inotropic effect, as well as the anti-cancer and anti-viral effects, are largely a result of the cardiac steroids-induced signaling activation. Indeed, the inhibition of ERK activation totally prevented the bufalin and other cardiac steroids-induced increase in heart contractility: the bufalin anti-cancer effect was shown repeatedly to be mediated by ERK and AKT signaling, as was the anti-viral activity of cardiac steroids. It is reasonable to suggest, therefore, that differences in cardiac steroids-induced signaling by various cardiac steroids has a profound effect on their pharmacological profiles.

Thus, compounds of the invention may be used for treatment or prevention of a disease or disorder in a subject, wherein the disease or disorder is optionally associated with activity of plasma membrane Na⁺/K⁺ ATPase, such that upon binding to this cationic pump, the steroids reduce the intracellular concentration of K⁺ while augmenting that of Nat. Diseases and disorders that are associated with plasma membrane Na⁺/K⁺ ATPase include a great variety of different types of heart diseases as well as cancers, neurological conditions as well as viral diseases. Thus, by diminishing or arresting activity of plasma membrane Na⁺/K⁺ ATPase, a variety of diseases including cardiac diseases, cancers, neurological conditions and viral diseases may be prevented or treated.

Thus, in another aspect, there is provided a compound of the invention for use in a method of preventing or reducing activity of plasma membrane Na⁺/K⁺ ATPase.

Maintaining proper Na⁺ and K⁺ gradients across a cell plasma membrane is an essential process for mammalian cell survival. It has been previously demonstrated that upon binding of cardiac steroids to Na,K-ATPase, at nontoxic doses, the role of the enzyme as a receptor is activated and intracellular signaling is triggered, upon which cancer cell death occurs. The Na,K-ATPase is also a key scaffolding protein that is able to interact with signaling proteins such as protein kinase C (PKC) and phosphoinositide 3-kinase (PI3K) and also works as a classical receptor in which the binding of cardiac glycosides induces activation of the tyrosine kinase Src and down-stream signaling cascades, independent of changes in intracellular ions. Viruses are very frequent causative agents of human infectious diseases and cancer, and their treatment is often challenging. Targeting host cell components such as Na,K-ATPase is a very promising antiviral strategy in order to minimize the resistance to antiviral drugs and has been shown to be effective in a broad spectrum of viral species.

Furthermore, Na,K-ATPase is a crucial protein responsible for the electrochemical gradient across the cell membranes. It regulates the entry of K+ with the exit of Na+ from cells, thereby maintaining Na+/K+ equilibrium through neuronal membranes. Oxidative metabolism is very active in brain, where large amounts of chemical energy as ATP molecules are consumed, essential for the maintenance of ionic gradients that underlie resting and action potentials and which are involved in nerve impulse propagation, neurotransmitter release and cation homeostasis. Evidence has supported the Na, K-ATPase involvement in signaling pathways, enzyme changes in diverse neurological diseases as well as during aging, in ischemia, brain injury, depression and mood disorders, mania, stress, Alzheimer's disease, learning and memory, and neuronal hyperexcitability and epilepsy.

Thus, compounds of the invention, with their improved performance, may be also used in the treatment or prevention of cancer, or may be used as anticancer drugs, alone or in combination therapy: may be used in the treatment or prevention of a viral infection or used as antiviral agents: or may be used as CNS agents for preventing a myriad of neurological conditions.

In some embodiments, compounds of the invention may be used in a method of preventing or treating a cardiac condition such as a heart failure and atrial fibrillation as well as fetal tachycardia, supraventricular tachycardia, cor pulmonale, and pulmonary hypertension.

In some embodiments, compounds of the invention may be used in a method of preventing or treating heart failure, atrial fibrillation, fetal tachycardia, supraventricular tachycardia, cor pulmonale, or pulmonary hypertension.

In some embodiments, compounds of the invention may be used for treatment or prevention of a disease or disorder associated with activation of intracellular signaling specifically the phosphorylation of ERK and AKT.

In some embodiments, compounds of the invention may also be used as anticancer agents for treating different types of carcinoma and other types of cancer.

In some embodiments, compounds of the invention may also be used as anti-viral compounds for preventing or treating viral infections or viral diseases.

In some embodiments, compounds of the invention may be used for treatment of different neurological or psychiatric disorders such as ischemia, brain injury, depression and mood disorders, bipolar disorder, mania, stress, Alzheimer's disease, learning and memory, and neuronal hyperexcitability and epilepsy.

Thus, the invention further provides use of compounds of the invention as Na, K-ATPase inhibitors and ERK and AKT stimulation.

The invention also provides a Na, K-ATPase inhibitor in the form of a compound of the invention.

In another aspect, the invention provides a composition comprising at least one compound according to the invention. In some embodiments, the composition is a pharmaceutical composition.

In some embodiments, the composition comprises at least one compound of the invention and at least one pharmaceutically acceptable carrier or excipient.

As acceptable in the art, cardiac steroids are administered in a variety of forms and a variety of administration modes. A composition according to the invention may be formed in a form suitable for oral administration, aerosol, parenteral, subcutaneous, intravenous, intramuscular, intraperitoneal, rectal, vaginal or topical administration.

Formulations suitable for oral administration can consist of (a) liquid solutions, such as an effective amount of the compound dissolved in diluents, such as water, saline, or orange juice: (b) capsules, sachets, tablets, lozenges, and troches, each containing a predetermined amount of the active ingredient, as solids or granules: (c) powders: (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Compounds of the invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer

Compositions suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The compounds can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

Compounds of the present invention may be made into injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

In another aspect, the invention provides a method of treatment or prophylaxis of a disease or disorder associated with the function of Nat, K⁺-ATPase, the method comprising administering to a subject (human or non-human) in need thereof an effective amount of a compound according to the invention.

The term treatment as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The effective amount for purposes herein is determined by such considerations as may be known in the art. The amount must be effective to achieve the desired effect as described above, i.e. inhibition of Na⁺, K⁺-ATPase, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

In some embodiments, compounds of the invention may be used in the treatment or prevention of a disease or disorder associated with inhibition of Na, K-ATPase cationic pump.

In some embodiments, compounds of the invention may be used in the treatment or prevention of a disease or disorder selected from heart diseases, such as heart failure, atrial fibrillation, fetal tachycardia, supraventricular tachycardia, cor pulmonale and pulmonary hypertension.

In some embodiments, compounds of the invention may be used in the treatment or prevention of cancer.

When used to fight a malignant proliferative disease or disorder, compounds of the invention can be used to treat a wide spectrum of cancers (neoplasms), such as blastoma, carcinoma, lymphoma, leukemia, sarcoma, mesothelioma, glioma, germinoma, choriocarcinoma, melanoma, glioblastoma, lymphoid malignancies and any other neoplastic disease or disorder, collectively referred to cancer.

Non-limiting examples of cancer which can be treated using compounds according to the invention include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments, compounds of the invention may be used in the treatment or prevention of a disease or disorder selected from neurological (Alzheimer's disease, Parkinson disease, ALS and other neurodegenerative disorders) and psychiatric disorders (such as depression, bipolar disorder, schizophrenia).

In some embodiments, compounds of the invention may also be used in prevention or treatment of viral diseases. Within the context of the invention, prevention or treatment of a viral infection encompasses preventing or minimizing replication of the virus in the cells, preventing enhancement of systemic immune functions, inhibition of viral titer increases in the body and reduction in the incidence of relapse of the viral infection and symptoms associated therewith. The prevention of viral infection also encompasses the reduction in the likelihood of infection with the virus such that (e.g., healthy) subjects administered with the composition of the present invention have a significantly less chance or are less susceptible to be infected by the virus or suffer from a viral infection as compared to untreated subjects.

The herein described method can be effective against more than one virus species, type, subtype, or strain and may be active in more than one host species. Thus, compounds according to the present invention can be effective in preventing viral infection of more than one type of virus (e.g., corona virus and a virus from the adenoviridae family).

In some embodiments, the virus is selected from coronaviridae/coronavirus, orthomyxoviridae, paramyxoviridae, Coxsackie family of viruses and adenoviridae family.

In some embodiments, compounds of the invention are for use in preventing infection by coronavirus.

In some embodiments, the coronavirus is a COVID-19 causing pathogen.

In some embodiments, the COVID-19 causing pathogen is SARS-COV-2.

As disclosed herein, the “SARS-COV-2” encompasses SARS-COV-2 having mutations that may be found in the entire genome of SARS-COV-2 strains, e.g., in the 5′ UTR, ORF1ab polyprotein, intergenic region, envelope protein, matrix protein, intergenic region and nucleocapsid protein.

### The Invention Provides:

A compound of general formula (I):

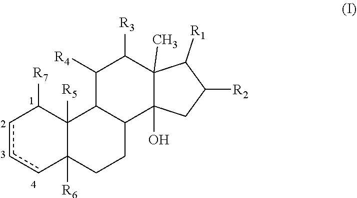

wherein
R₁ is a lactone,
each of R₂, R₃, R₄, R₅, R₆ and R₇, independently of the other, is selected from —H, —OR′, —C₁-C₅alkyl, —C₁-C₅alkylene-hydroxyl and —C(—O)R″,
each of R′ and R″, independently of the other, is selected from —H, —C₁-C₅alkyl, —C₁-C₅alkylene-hydroxyl and —C(—O)C₁-C₅alkyl; and
a bond designated [symbol] is a double bond or a single bond, such that one of the bonds designated [symbol] is a double bond and the other is a single bond.

In some configurations of a compound of the invention, the lactone is a 5-memebred lactone ring or a 6-memebered lactone ring.

In some configurations of a compound of the invention, the lactone ring comprises one or two endocyclic double bonds.

In some configurations of a compound of the invention, the lactone is lactone (i) or lactone (ii):

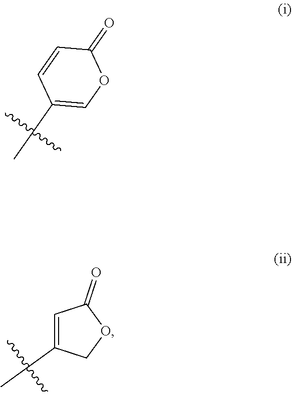 [SMILES: *c1ccc(=O)oc1 ; *C1=CC(=O)OC1]

wherein [symbol] designates a bond of connectivity.

In some configurations of a compound of the invention, the compound is a compound of the general formulae (Ia) or (Ib)

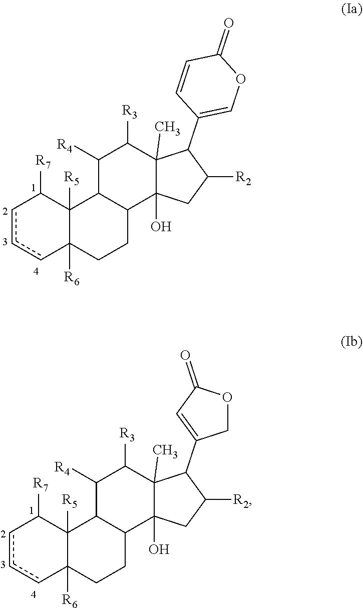

wherein each of each of R₂, R₃, R₄, R₅, R₆, R₇, R′, R″ and [symbol] is as defined herein.

In some configurations of a compound of the invention, R₂ is selected from —H and —OR′, wherein R′ is as defined herein.

In some configurations of a compound of the invention, R′ is —H.

In some configurations of a compound of the invention, R′ is —C(═O)C₁-C₅alkyl.

In some configurations of a compound of the invention, —C(═O) C₁-C₅alkyl is —C(═O)CH₃.

In some configurations of a compound of the invention, R₃ is selected from —H and —OH.

In some configurations of a compound of the invention, R₃ is —H.

In some configurations of a compound of the invention, R₄ is selected from —H and —OH.

In some configurations of a compound of the invention, R₄ is —H.

In some configurations of a compound of the invention, R₅ is selected from —H, —C₁-C₅alkyl, —C₁-C₅alkylene-hydroxyl and —C(═O)R′, wherein R′ is selected from —H and —C₁-C₅alkyl.

In some configurations of a compound of the invention, R₅ is —C₁-C₅alkyl.

In some configurations of a compound of the invention, —C₁-C₅alkyl is —CH₃.

In some configurations of a compound of the invention, R₆ is selected from —H and —OH.

In some configurations of a compound of the invention, R₆ is —H.

In some configurations of a compound of the invention, R₇ is selected from —H and —OR′, wherein R′ is selected from —H and —C₁-C₅alkyl.

In some configurations of a compound of the invention, R₇ is —H.

In some configurations of a compound of the invention, the bond between C3 and C2 is a double bond.

In some configurations of a compound of the invention, the bond between C3 and C4 is a double bond.

In some configurations of a compound of the invention, each of R₂, R₃, R₄, R₆ and R₇ is —H and R₅ is —CH₃.

In some configurations of a compound of the invention, bond between C3 and C2 is a double bond.

In some configurations of a compound of the invention, bond between C3 and C4 is a double bond.

A compound having structure (IIa) or (IIb):

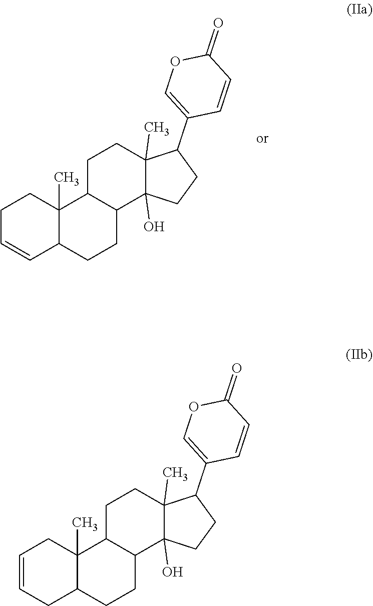 [SMILES: CC12CCC=CC1CCC1C2CCC2(C)C(c3ccc(=O)oc3)CCC12O ; CC12CC=CCC1CCC1C2CCC2(C)C(c3ccc(=O)oc3)CCC12O]

Use of a compound according to the invention, for use in medicine.

In some configurations of a compound of the invention, for use in a method of treating a disease or a disorder in a subject.

In some configurations of a compound of the invention, for use in treating a disease or disorder that is cancer, or a viral infection, or a disease or disorder that is associated with Na⁺/K⁺ ATPase activity, or a disease or disorder that is selected from heart diseases, cancers, neurological diseases and psychiatric diseases.

In some configurations of a compound of the invention, for use in treating or preventing a heart disease that is selected from heart failure, atrial fibrillation, fetal tachycardia, supraventricular tachycardia, cor pulmonale and pulmonary hypertension.

A composition comprising a compound of the invention.

In some configurations of a composition of the invention, the composition is a pharmaceutical composition.

In some configurations of a composition of the invention, for use in treatment or prevention of a disease or disorder.

In some configurations of a composition of the invention, for use in treatment or prevention of a disease or disorder associated with Na⁺/K⁺ ATPase activity.

In some configurations of a composition of the invention, wherein the disease or disorder is selected from heart diseases, cancers, neurological diseases and psychiatric diseases.

In some configurations of a composition of the invention, wherein the heart disease is selected from heart failure, atrial fibrillation, fetal tachycardia, supraventricular tachycardia, cor pulmonale and pulmonary hypertension.

In some configurations of a composition of the invention, for use in preventing or treating a viral infection.

In some configurations of a composition of the invention, wherein the infection is by a coronavirus.

In some configurations of a composition of the invention, wherein the coronavirus is a COVID-19 causing pathogen.

In some configurations of a composition of the invention, wherein the COVID-19 causing pathogen is SARS-COV-2.

In some configurations of a composition of the invention, the composition comprising at least one pharmaceutically acceptable carrier or excipient.

In some configurations of a composition of the invention, in a from selected from tablets, capsules, liquids, vials, IV bags, ampoules, cartridges, prefilled syringes, eye drops, nasal drops, nebulizers and inhalers, creams, ointments, gels, lotions, vaginal suppositories, rectal suppositories and enemas.

In some configurations of a composition of the invention, in a form suitable for oral, aerosol, parenteral, subcutaneous, intravenous, intramuscular, intraperitoneal, rectal, vaginal or topical administration.

A method of treatment or prophylaxis of a disease or disorder associated with with Na⁺/K⁺ ATPase activity, the method comprising administering to a subject (human or non-human) in need thereof an effective amount of a compound according to the invention.

In some configurations of a method of the invention, wherein the disease or disorder is selected from heart failure, atrial fibrillation, fetal tachycardia, supraventricular tachycardia, cor pulmonale, pulmonary hypertension, cancers, neurological diseases and psychiatric diseases.

A method of treatment or prophylaxis of cancer, the method comprising administering to a subject (human or non-human) in need thereof an effective amount of a compound according to the invention.

A method of treatment or prophylaxis of a disease or disorder associated with a viral infection, the method comprising administering to a subject (human or non-human) in need thereof an effective amount of a compound according to the invention.

In some configurations of a composition of the invention, wherein the infection is caused by a coronavirus.

In some configurations of a composition of the invention, wherein the coronavirus is a COVID-19 causing pathogen.

In some configurations of a composition of the invention, wherein the COVID-19 causing pathogen is SARS-COV-2.

A de-hydroxy cardiac steroid as defined herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the X-Ray crystallography of bufalin dehydroxy-3,4-ene (2).

FIG. 2 depicts effect of Compound 1 on Brain Microsomal Na⁺, K⁺-ATPase activity. Na⁺, K⁺-ATPase activity in rat brain microsomal fraction was determined as previously described (Lichtstein, D et al. Hypertension 7:729-733, 1985) by the colorimetric determination of inorganic phosphate after the incubation of microsomes (30 μg protein/reaction) at 37° C. in a solution (final volume 500 μl) containing (final concentrations): Tris-HCl (50 mM, pH 7-4), NaCl (100 mM), KCl (10 mM), MgCl₂ (4 mM) and ATP (2 mM, Tris, vanadium free). After 10-min pre-incubation, the ATP was added to initiate the reaction. Reactions were terminated by the addition of 100 μl 5% trichloroacetic acid and the precipitate was removed by centrifugation.

FIG. 3 depicts the effect of Compound 2 on brain Microsomal Na⁺, K⁺-ATPase activity.

FIG. 4 depicts the effect of Acetylcholine and Norepinephrine on Cardiomyocytes Contractility.

FIG. 5 depicts the effects of Digoxin and New Synthetic-Steroids on Cardiomyocytes Contractility (10 minutes).

FIG. 6 depicts the effects of Digoxin and New Synthetic-Steroids on Cardiomyocytes Contractility (30 minutes).

FIG. 7. depicts the effects of compound 1 on SARS-Cov-2 viral infection and toxicity in vero cells.

FIG. 8 depicts the effects of compound 2 on SARS-Cov-2 viral infection and toxicity in vero cells.

FIG. 9 depicts the effects of bufalin on SARS-Cov-2 viral infection and toxicity in vero cells.

FIG. 10 depicts the effects of digoxin on SARS-Cov-2 viral infection and toxicity in vero cells.

FIG. 11 depicts the effects of ouabain on SARS-Cov-2 viral infection and toxicity in vero cells.

FIG. 12 depicts the effects of remdesivir on SARS-Cov-2 viral infection and toxicity in vero cells.

FIG. 13 depicts the effects of bufalin on growth of different cancer cell.

FIG. 14 depicts the effects of compound 1 on growth of different cancer cells.

FIG. 15 depicts the effects of compound 2 on growth of different cancer cells.

### DETAILED DESCRIPTION OF EMBODIMENTS

Bufalin, the major component of the traditional Chinese medicine Chan-Su, is an extract of the skin and parotid venom glands of a toad of the Bufo family. Chan-Su has been widely used in China and other Asian countries to treat cancer and additional ailments. Bufalin belongs to the cardiac steroid family and, like other members such as ouabain and digoxin, increases the force of contraction of heart muscle, thus improving circulation in cases of insufficient cardiac output. However, the toxicity and the small therapeutic window of this family of steroids limits their use as cardiotonic drugs. A similar problem is encountered in the use of these steroids for the treatment of cancer: Although bufalin has been shown to kill various tumor cells in vitro, it produced unsatisfactory results when administered in vivo. Because of its fast metabolism, toxicity, insolubility in water and short half-life, its application in the clinical setting is limited. In addition, bufalin and other cardiac steroids have been shown to have potent anti-inflammatory and anti-viral activities. However, all these beneficial properties are obtained at concentrations higher than the toxic effects of these compounds. Therefore, the development of bufalin derivatives with lower toxicity is of great importance.

The plasma membrane Na⁺ and K⁺ transporter Nat, K⁺-ATPase is an established receptor for cardiac steroids. The interaction of these steroids with Na⁺, K⁺-ATPase results in inhibition of the ion-pumping function and, in addition, causes the activation of several signal transduction cascades, including mitogen-activated protein kinase: extracellular signal-regulated kinase: proto-oncogene tyrosine-protein kinase (Src): PI3K/Akt, Ca⁺⁺ signaling, and reactive oxygen species generation pathways. It is well established that the toxicity of cardiac steroids in the heart is due to calcium overload, produced by excessive inhibition of the Nat, K⁺-ATPase in the myocytes, leading to arrhythmia and lethality. Conversely, the positive inotropic effect, as well as the anti-cancer and anti-viral effects, are largely a result of the CS-induced signaling activation. Indeed, the inhibition of ERK activation totally prevented the bufalin and other CS-induced increase in heart contractility: the bufalin anti-cancer effect was shown repeatedly to be mediated by ERK and AKT signaling, as was the anti-viral activity of cardiac steroids. It is reasonable to suggest, therefore, that differences in cardiac steroids-induced signaling by various CS will have a profound effect on their pharmacological profiles.

Cardiac steroids are composed of three major structural components: a steroid core, in which rings AB and CD are cis-fused, whereas rings BC are trans-fused: a 5- and 6-membered lactone ring at position 17 (cardenolides and bufadienolides, respectively); and a variable number of sugar residues at position 3. The significance of the moiety orientation at position 3 of the steroid for its biological activity was studied extensively, especially in relation to the nature of the sugar bound at this position. Furthermore, the inventors' previous study showed that the a/B orientation of the 3-OH group may have a substantial effect on biological activity. Whereas the 3-OH β isomer displayed the standard capability of increasing heart contractility, an a 3-OH isomer did not boost the force of contraction, but actually inhibited the contractility induced by digoxin.

In the present application, two isomers lacking an OH at the 3 position, bufalin-2,3-ene and bufalin-3,4-ene, were synthesized and studied as examples of de-hydroxylated cardiac steroids. The biological activities of these compounds were evaluated by testing their ability to inhibit Nat, K⁺-ATPase activity in a pig brain microsomal fraction, to induce ERK and AKT phosphorylation in human neuroblastoma LAN-5 cells, to cause cytotoxicity in human cancer cells and to prompt positive inotropy in quail cardiac cells in culture.

### Materials and Methods

### Materials

Solvents were purchased from Romical (Jerusalem, Israel), bufalin and Ishikawa's reagent were purchased from Chengdu Biopurify Phytochemicals Ltd. Wenjiang, Chengdu, China and Sigma Aldrich Co. (St. Louis MO, USA), respectively. TLC Silica Gel 60 F254 Aluminum Sheets were purchased from Merck, (Darmstadt, Germany) and Sep-pak, C18 columns from Waters (Milford, MA, USA). A549, alveolar basal epithelia cell carcinoma, HCT 116, colonorectal carcinoma, and HFF-1, human fibroblasts were obtained from ATCC, (Manassas, VA. USA). HaCaT, immortalized keratinocytes were obtained from AddexoBio (San Diego, CA, USA) and U251 glioblastoma cells were from ECACC General Cell Collection (Salisbury, United Kingdom). Serum, DMEM cell culture medium, antibiotics and a chemiluminescence kit were acquired from Biological Industries (Beit Ha emek, Israel). ATP and protease inhibitor cocktail were purchased from Sigma-Aldrich, (St. Louis, MO, USA). Horseradish peroxidase-conjugated secondary antibodies were purchased from Jackson ImmunoResearch Laboratories, West Grove, PA, USA. Bradford reagent and Laemmli sample buffer were provided by Bio-Rad Laboratories, Hercules, CA, USA. Polyvinylidene fluoride membranes were purchased from Pall Corporation, Pensacola, FL, USA and a Pierce primary cardiomyocyte isolation kit was obtained from Thermo Scientific, Rockford, Il, USA.

### High Performance Liquid Chromatography

HPLC was performed with a Hewlett-Packard (HP) 1050 Series chromatograph with a HP 1010 detection system and an Agilent computer system. A 50 μl volume of each sample was passed through a Luna C-18, 5 μm column (250×4.6 mm) Phenomenex, Torrance, CA, USA), provided with a pre-column. Elution was performed with a 35 min, 68% CH₃CN/water isocratic system with a flow rate of 1.0 ml/min.

### NMR and Mass Spectroscopy

The 1H NMR (500 MHZ) measurements were performed with a Bruker AVANCE III HD 500 Mhz spectrometer in CDCl3. Electron spray mass spectra were obtained with a Quadrupole LCMS mass spectrometer system (Thermo Scientific, USA).

### Crystallographic Structure Analysis

Crystallography was performed on an ENRAF-NONIUS CAD-4 computer-controlled diffractometer, and all crystallographic computing was made with a VAX9000 computer at the Hebrew University of Jerusalem.

### ATPase Activity

A pig brain microsomal fraction was prepared as previously described. Na⁺, K⁺-ATPase activity in the microsomal fraction was determined by the amount of inorganic phosphate released during incubation at 37° C. In brief: 480 μl of the microsomal preparation (60 μg protein) was added to 3520 μl reaction buffer (50 mM Tris-Base, 120 mM NaCl, 10 mM KCl, 4 mM MgCl₂, pH 7.4) in the presence of varying concentrations of an inhibitor (bufalin or bufalin derivatives). Following 20 min. incubation, 10 μl of ATP (2.5 mM final concentration) was added and the incubation was allowed to proceed for an additional 30 min. The reaction was terminated by the addition of 1 ml 16% trichloroacetic acid and placing the tubes on ice for 10 min. Following centrifugation (500×g, 10 min, 4° C.), 50 μl of the supernatant was removed for determination of inorganic phosphate according to a colorimetric method, as described previously.

### ERK and AKT Phosphorylation in LAN-5 Cells:

Human neuroblatoma LAN-5 cells were grown in RPMI 1640 supplemented with penicillin-streptomycin (PS) (100×)-1%, l-glutamine (100×) 200 mM-1%, and FBS-10%-50 ml, in an incubator maintained at 37° C., with 5% CO₂. Before starting the experiment, the cells were transferred to 6 well plates at a density of 100,000 cells/well and grown for 24 hrs in serum-free medium. The steroids (10 nM final concentration) were then added and the wells were incubated for 10 min. The proteins were then extracted by adding RIPA lysis buffer and protease inhibitor (1:100) and centrifuged (14,000×g). The protein content of the supernatants was determined according to Bradford. Then the supernatants were diluted in Laemmli sample buffer and incubated at 95° C. for 5 min. A total 30 μg of each sample was loaded onto a 12% sodium dodecyl sulfate polyacrylamide gel and electrophoresed. The proteins were transferred to polyvinylidene fluoride membranes which were blocked with Tris-buffered saline containing 0.1% (v/v) Tween and 5% (w/v) skim milk and incubated overnight at 4° C. with PathScan Multiplex Western Cocktail I containing phospho-p44/42 (ERK1/2 Tyr204) (D13.14.4E) XP® rabbit mAb for ERK, and Phospho-Akt (Ser473) (D9E) XPR rabbit mAb for AKT. The membranes were then incubated with horseradish peroxidase-conjugated secondary antibodies (Jackson ImmunoResearch Laboratories, West Grove, PA, USA). Antibodies were detected with an enhanced chemiluminescence kit, according to the manufacturer's instructions. Signals were visualized on film (Kodak: BioMax, Wellsville, NY, USA) and quantified densitometrically (Fluro-s Multilmager: Bio-Rad, Hercules, CA, USA).

### Cytotoxicity Against Cancer and Normal Cells

The anti-proliferative activity of the target compounds was tested against the NCI-60 cell line panel. The screening was performed at the National Cancer Institute (NCI), Bethesda, Maryland, USA (www.dtp.nci.nih.gov), according to their standard protocol (https://dtp.cancer.gov/discovery_development/nci-60/methodology.htm).

All cell lines were grown at at 37° C. in 5% CO₂. A549 cell line were grown in F-12 (Ham's) media with L-Glutamine supplemented with 10% FBS, 1% sodium pyruvate and 1% Penicillin-Streptomycin. HCT 116 cell line were grown in McCoy's 5A Medium with L-Glutamine, supplemented with 10% FBS, 1% sodium pyruvate and 1% Penicillin-Streptomycin. U-251 cell line were grown in MEM-NEAA, Earle's Salts Base, with Non-Essential Amino Acids, supplemented with 10% FBS, 1% sodium pyruvate, 1% L-Alanyl-L-Glutamine, 0.01 mg/ml human recombinant insulin and 1% Penicillin-Streptomycin. HaCaT and HFF-1 cell lines were grown in DMEM with L-Glutamine, supplemented with 10% FBS, 1% sodium pyruvate and 1% Penicillin-Streptomycin.

One day before the experiments cells from all cell lines were seeded into 96-well plates at a density of 1×10⁴ cells/well in 100 μl of appropriate growth media. Plates were incubated overnight in 37° C. to allow attachment. Growth media (100 μl) containing the tested steroids were added to obtain the desired concentration and the cells were incubated for 48 hours in 37° C. Cells incubated in growth media containing 0.33% DMSO (vehicle) served as control. At the end of incubation period, cell viability evaluation was performed using VisionBlue™ Quick Cell Viability Fluorometric Assay Kit, according to manufacturer's instructions. Fluorescent signal by was detected by TECAN spark 10M microplate reader, Excitation/Emission 535±25/590±20 nm. The fluorescence data is expressed as percentage of cell viability (%) compared to vehicle control.

### Quail Cardiac Muscle Cell Contractility

Quantification of cardiomyocytes contractility was performed as previously described. Since avian embryos (quail included) are not considered “animals”, their use is exempt at the Hebrew University, like in all other academic institutions, of the need for ethical approval. Quail (Coturnix japonica) cardiomyocytes were prepared from E4 embryos with a Pierce Primary Cardiomyocyte Isolation Kit (Thermo Scientific™). Contractility at 37° C. was measured 30 min after drug addition. Cells were photographed for 15 sec, with an Olympus CKX41 (Japan) upright microscope (×20 magnification), and integrated incandescent illumination. A FastCam imi-tech (Korea) high speed digital camera with a 640×480 pixel gray scale image sensor was mounted on the microscope with ImCam software (IMI Tecnology, Co. Ltd Gangnam-gu, Seoul, South Korea). Changes in cell contraction were deduced from the mean difference in area change between relaxation and the contraction peaks. Three cell clusters in 3 wells were photographed and measured under each experimental condition.

### Statistical Analysis

All the data are expressed as the mean±standard error (SEM). Significance was determined according to the independent Student's t-test: p<0.05 was considered significant.

### The Synthesis of Novel Dehydroxy Bufalin

Ishikawa's reagent ((CH₃CH₂)₂NCF₂CHFCF₃) was used to produce the new compounds.

### Experimental:

### Synthesis

In a 2 ml glass vial provided with a magnetic stirrer a suspension of bufalin (15 mg) in diethylether (1.0 ml was prepared. The vial was cooled at 10 C, wrapped in Aluminium foil and Ishikava's reagent (35-40 mg, 50-60 μl) was slowly added. The mixture was stirred overnight at room temperature. Next day, the product was monitored by TLC and HPLC for the new peaks that were formed (RT 26-28 min). In order to separate the product from the excess of the reagent, the solution was loaded on a silica gel TLC plate and eluted with 70% diethyl ether and hexane for 4 min. Following visualization with a UV lamp at 260 nm, the silica gel plate at 2 cm was scraped into a small vial and the organic material dissolved in methanol (0.3 ml). From the filtrated solution, 50 μl was injected in to the HPLC system. Two products (at RT 27 and 28.5) were detected. The first product bufalin 2,3-ene is herein designated Compound 1 and the second product bufalin 3,4-ene is herein designated Compound 2.

HPLC was performed with a Hewlett-Packard (HP) 1050 Series chromatograph provided with a HP 1010 detection system and an Agilent ChemStation (Waldbron, Germany). The detector was set to 220 and 260 nm and the sample was injected to a Luna C-18, 5 μm column (250×4.6 mm) Phenomenex, Torrance, CA) provided with a pre-column. The elution was performed with a 35 min CH₃CN/water system with a flow rate of 1.0 ml/min. 50 μl of sample was injected into HPLC, the elution was carried out with 68% CH₃CN in water.

The Electron spray mass spectrum was obtained using a Quadrupole LCMS mass spectrometer system (Thermo scientific, USA).

The two compounds were collected, dried, and subjected to mass spectra, NMR and X Ray crystallographic measurements. The first product Compound 1, appears an amorphous solid powder and the second solid Compound 2, formed large crystals.

Both compounds showed identical mass spectrum with a protonated molecular mass at 369 that can correspond to isomers of dehydroxy bufalin formed by elimination of one of the OH groups from either C3 or C14 position with a hydrogen atom from a nearby position (bufalin —H₂O).

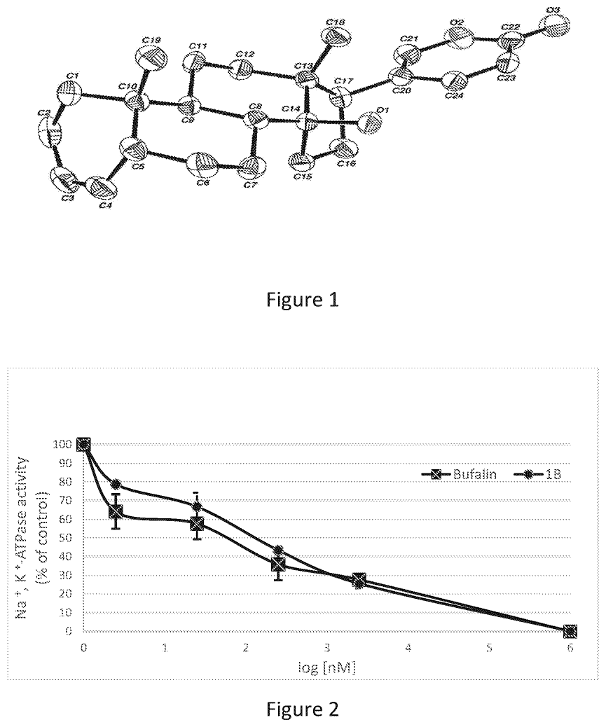

Compound 2 was recrystallized from acetonitrile/water 70% and subjected to X ray crystallography measurements that produced the unexpected structure, bufalin dehydroxy-3,4-ene (FIG. 1). In other words, suggesting an unusual catalytic effect induced by the Ishikawa's reagent to produce the elimination of the 30H group leading selectively to a double bounds in ring A, the dehydroxy bufalin isomers are shown below:

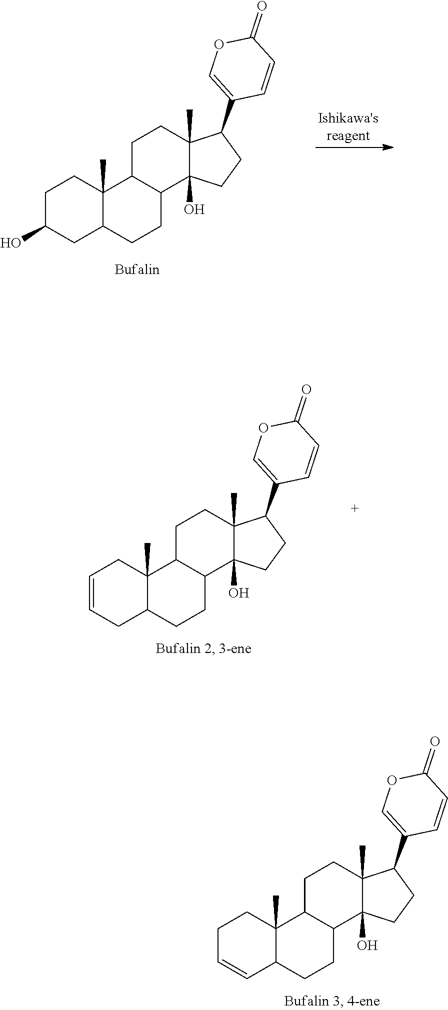 [SMILES: C[C@]12CC[C@H](O)CC1CCC1C2CC[C@]2(C)[C@@H](c3ccc(=O)oc3)CC[C@]12O ; C[C@]12CC=CCC1CCC1C2CC[C@]2(C)[C@@H](c3ccc(=O)oc3)CC[C@]12O ; C[C@]12CCC=CC1CCC1C2CC[C@]2(C)[C@@H](c3ccc(=O)oc3)CC[C@]12O]

Examination of the proton NMR of compound 1 showed in addition to the vinyl hydrogens of the diolone ring, two symmetrical vinyl hydrogens at 5.3 and 5.5 ppm, corresponding to a bufalin dehydroxy-2,3-ene isomer. Further, examination of NMR spectrum of the 1:2 mixture of Compound 1 and Compound 2 isomers shows clearly the doublets of the vinyl hydrogens at 5.4 and 5.7 ppm, corresponding the 3,4-ene double bound as an additional prove of the structure of Compound 2.

### Biological Effects

Na⁺, K⁺-ATPase Activity Inhibition

The only established receptor for cardiac steroids including bufalin is the plasma membrane enzyme, the Nat, K⁺-ATPase. The inhibition of Na⁺, K⁺-ATPase activity by bufalin is the underlying mechanism for the increase in force of contraction of heart muscle as well as other biological effects of the steroid (for review see Clausen M V. Et a1. Front Physiol. 2017: Schoner W and Scheiner-Bobis G. Am J Physiol. Cell Physiol. 293: C509-536, 2007). The a2 and a3 isoforms of the Nat, K⁺-ATPase are more sensitive to the cardiac steroids and are those that participate in many of their pharmacological effects. Hence, the ability of novel bufalin derivatives to inhibit Na⁺, K⁺-ATPase activity is a measure for their potency to increase cardiac contractility and affect other biological processes (Blaustein M P. Am J Physiol. 309: H958-968, 2015; Yuen G K et al. J Mol. Cell Cardiology 108:158-169, 2017).

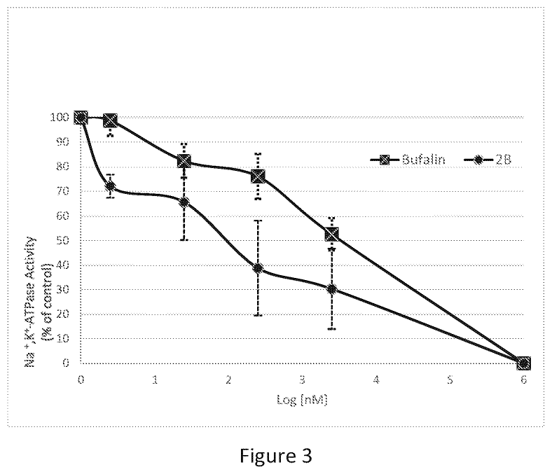

The inhibition of brain microsomal Na⁺, K⁺-ATPase activity by Compounds 1 and 2 in comparison to that of Bufalin are depicted in FIGS. 2 and 3, respectively. It can be seen that while Compound 1 has approximately the same potency as Bufalin, Compound 2 is a more potent inhibitor. Importantly, Compound 2 inhibit Na⁺, K⁺-ATPase activity better than Bufalin even at the very low (nanomolar) concentrations, relevant to inhibitions of the a2 and a3 isoforms of the Na⁺, K⁺-ATPase.

Na⁺, K⁺-ATPase activity in rat brain microsomal fraction was determined as previously described (Lichtstein, D et al. Hypertension 7:729-733, 1985) by the colorimetric determination of inorganic phosphate after the incubation of microsomes (30 μg protein/reaction) at 37° C. in a solution (final volume 500 μl) containing (final concentrations): Tris-HCl (50 mM, pH 7-4), NaCl (100 mM), KCl (10 mM), MgCl₂ (4 mM) and ATP (2 mM, Tris, vanadium free). After 10-min pre-incubation, the ATP was added to initiate the reaction. Reactions were terminated by the addition of 100 μl 5% trichloroacetic acid and the precipitate was removed by centrifugation.

### Increase of Heart Cells Contractility

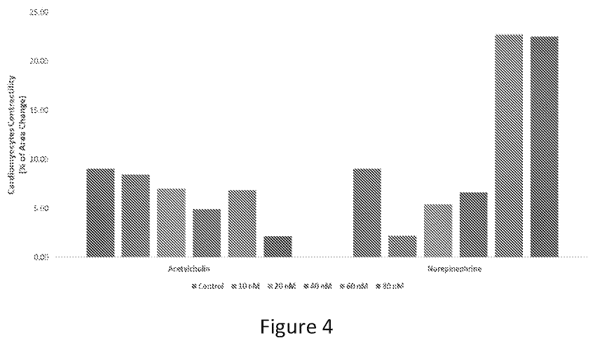

To test for the potential effect of the synthetic compounds on heart muscle contractility, this was examined in quail cardiomyocytes. Basic characterization of this system was performed by testing the effects of the classical neurotransmitters acetylcholine and nor-adrenalin cardiomyocyte's contractility. As can be seen in FIG. 4, as expected, acetylcholine and noradrenaline, dose-dependently, decreased and increased, respectively, cardiomyocytes contractility.

Quails cardiomyocytes were prepared from quail embryos at E4. Contractility at 37° C. was measured 10 minutes after drug addition. Cells were photographed for 15 seconds, and the change in cell contraction was deduced from the mean difference in area change between relaxation and the contraction peaks. 3 cell clusters in 3 wells were photographed and measured for each experimental condition. Quantification of motility is displayed as the cell Area Change.

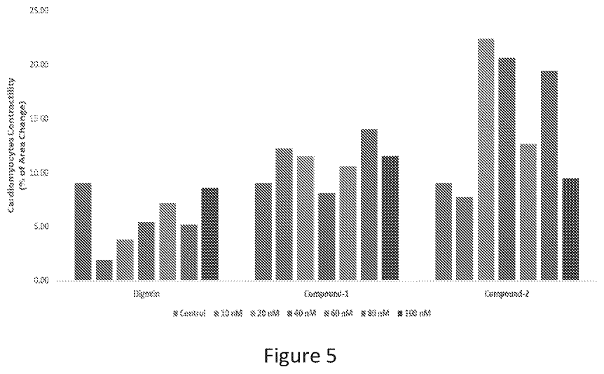

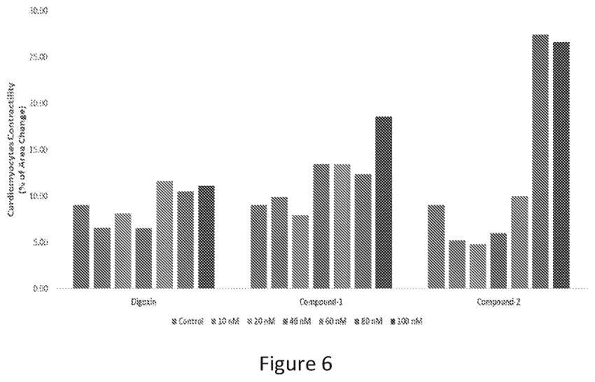

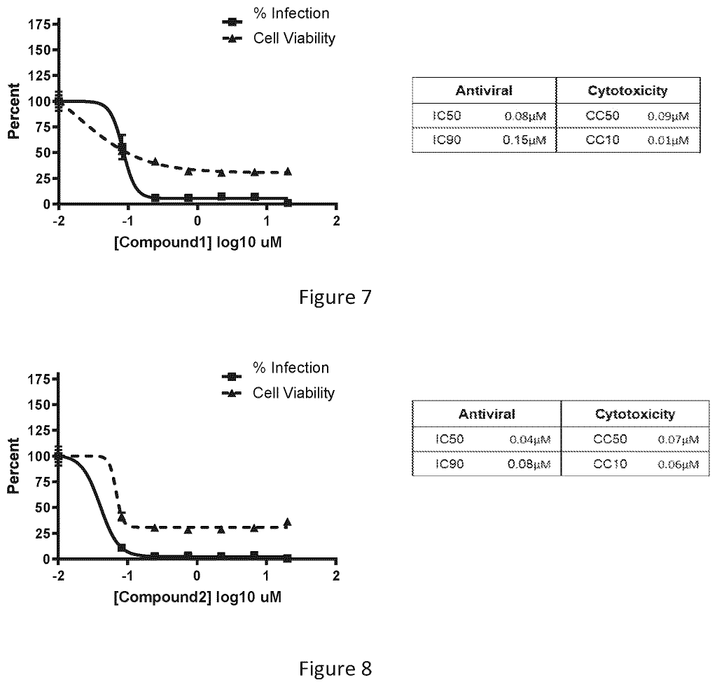

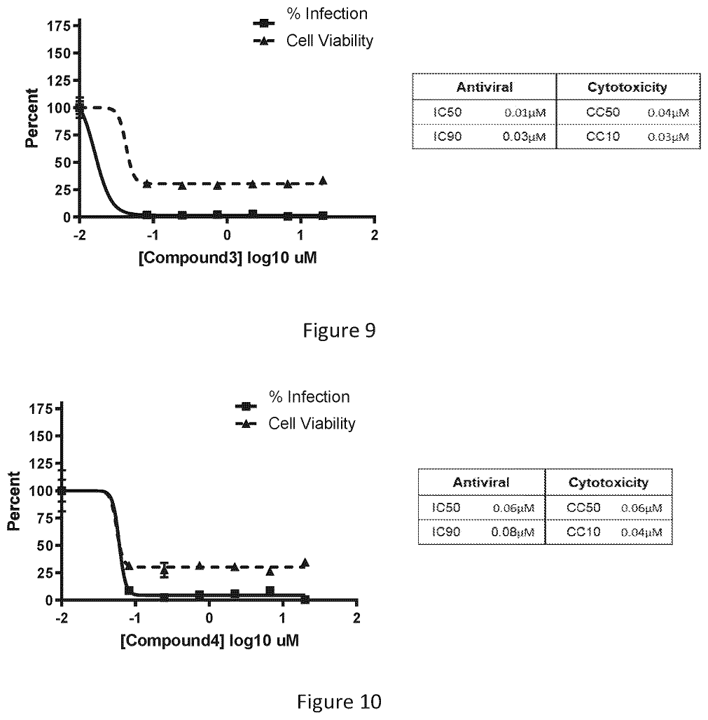

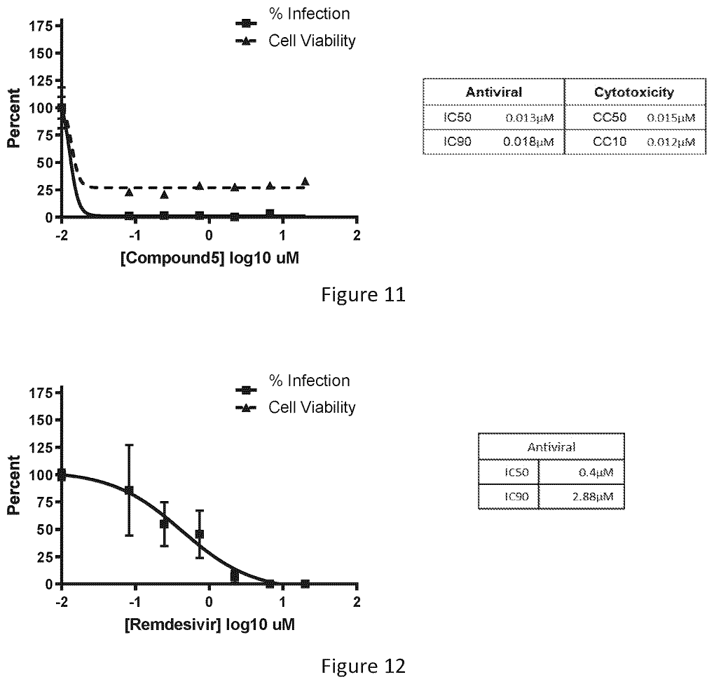

The effects of the cardiac steroid digoxin and the two new synthetic compounds on quail cardiomyocytes contractility is depicted in FIG. 5. Most notably, compound 1 and compound 2 increase dramatically cardiomyocytes contractility compared to the effect of the known steroid. At all concentrations tested, compound 1 and most so, compound 2 had a stronger effect on contractility than digoxin. This was seen when contractility was measured 10 minutes (FIG. 5) and 30 minutes (FIG. 6) after the steroids addition.

Quails cardiomyocytes were prepared from quail embryos at E4. Contractility at 37° C. was measured 10 minutes after drug addition. Cells were photographed for 15 seconds, and the change in cell contraction was deduced from the mean difference in area change between relaxation and the contraction peaks. 3 cell clusters in 3 wells were photographed and measured for each experimental condition. Quantification of motility is displayed as the cell Area Change. New synthetic steroids are: Bufalin deoxy-2,3-ene (Compound 1) and Bufalin deoxy-3,4-ene (Compound 2).

Cytotoxic Effect of the Synthetic Compounds in Comparison to that of Other Cardiac Steroids.

The effect of compound 1 and compound 2 on cell viability was determined using neuroblastoma SH-SY5Y cells. As seen in Table 1 below, ouabain, the most studied cardiac steroid, and digoxin, the cardiac steroid that used most frequently used in the clinic caused a complete cell death already at 5-25 μM. Digoxin, Importantly, both compounds 1 and 2 exhibit a lower cytotoxic potency at all concentrations tested, in comparison to the other steroids.

TABLE 1
Effect of Cardiac Steroids and New Synthetic Compounds
on Neuroblastoma (SH-SY5Y) cells Viability.

| Compound | Concentration (μM) | Cell Viability (%) |
|---|---|---|
| Control | — | 100 |
| Ouabain | 5 | 6.26 |
| | 25 | 1.92 |
| | 50 | 0.51 |
| | 100 | 4.09 |
| Digoxin | 5 | 23.37 |
| | 25 | 1.02 |
| | 50 | 0.26 |
| | 100 | 2.55 |
| Compound 1 | 5 | 32.21 |
| | 25 | 13.79 |
| | 50 | 5.62 |
| | 100 | 1.28 |
| Compound 2 | 5 | 53.83 |
| | 25 | 24.71 |
| | 50 | 11.49 |
| | 100 | 6.13 |

Neuroblastoma SH-SY5Y cells (ATCC, Manassas, VA, USA) were grown in 96 wells plate in DMEM and Ham's F12 growth media supplemented with 10% fetal calf serum containing 100 μg/ml streptomycin and 100 U/ml penicillin at 37° C. and 5% CO2. Cardiac steroids were added in a media without 10% fetal calf serum FCS and cell viability was measured after 16 hr. using the conventional MTT assay. MTT was dissolved in above mentioned media at a concentration of 5 mg/ml and 50 μL of the solution was added to each well and plates were incubated at 37° C. for 2 h. After incubation media was dispensed and 200 μL of DMSO was added to each well to dissolve the MTT formazan, with 30 min incubation at room temperature. Absorbance was measured with an ELISA-plate reader (Bio-Tek Instruments) at 570 nm to quantify the amount of formazan product, which reflects the number of viable cells in culture and the percent viability was calculated with respect to control untreated cells.

### Anti-Viral Activity of Compounds of the Invention

Recently, Na⁺, K⁺-ATPase a1 subunit mediated Src signaling was reported to be involved in viruse entry into cells (Burkard, C., Verheije, M. H., Haagmans, B. L., van Kuppeveld, F. J., Rottier, P. J., Bosch, B. J., de Haan, C. A., 2015. ATPIA1-mediated Src signaling inhibits coronavirus entry into host cells. J. Virol. 89, 4434-4448). This study showed that ouabain and bufalin at nM concentrations inhibited infection of cells with MHV, FIPV, Middle East respiratory syndrome (MERS)-CoV, and VSV, but not IAV.

In a recent study ouabain was found to diminish anti-transmissible gastroenteritis coronavirus (TGEV) activity in swine testicular (ST) cells titers and inhibit the TGEV-induced production of IL-6 in a dose dependent manner (Yang, C.W., Chang, H. Y., Hsu, H. Y., Lee, Y. Z., Chang, H. S., Chen, I. S., Lee, S. J., 2017a. identification of anti-viral activity of the cardenolides, Na+/K+-ATPase inhibitors, against porcine transmissible gastroenteritis virus. Toxicol. Appl. Pharmacol. 332, 129-137).

The effect of cardiac steroids on viral infection was also demonstrated for other viruses. Ouabain and other related steroids inhibited Human respiratory syncytial virus (RSV) entry into human small airway epithelial cells (Lingemann M. The alpha-1 subunit of the Na⁺, K⁺-ATPase (ATP1A1) is required for macropinocytic entry of respiratory syncytial virus (RSV) in human respiratory epithelial cells. PLOS Pathog 15 (8): e1007963, 2019). Treatment of human cells with digoxin or ouabain, resulted in a dose-dependent decrease in infection by Chikungunya virus (CHIKV) (Ashbrook A W et al. Antagonism of the Sodium-Potassium ATPase Impairs Chikungunya Virus Infection. mBio. 2016 7 (3)). The effect was cell type-specific, as the steroid treatment of either murine or mosquito cells did not diminish CHIKV infection. Screening for anti-Japanese Encephalitis Virus infection identified ouabain and digoxin having robust efficiency against the virus (Guo J. et al. Screening of natural extracts for inhibitors against Japanese Encephalitis Virus infection. Antimicrob Agents Chemother 64: e02373-19). Ouabain was shown to impair herpes simplex virus infection. In contrast to the effects described above, in this study, the steroid did not inhibit viral attachment or entry, but did affect replication, reducing the expression of viral immediate-early and early genes by at least 5-fold (Dodson A W. Et al. Inhibitors of the sodium potassium ATPase that impair herpes simplex virus replication identified via a chemical screening approach. Virology 366 (2007) 340-348). Recently it was demonstrated that ouabain decrease influenza virus replication by inhibiting cell protein translational machinery (Amarella L. et al. Cardiac glycosides decrease influenza virus replication by inhibiting cell protein translational machinery. Am J Physiol Lung Cell Mol Physiol. 2019 Jun. 1; 316 (6): L1094-L1106).

### Experimental

Vero E6 cells were pre-treated for 2 h with increasing concentrations of the indicated compound and then infected with SARS-COV-2. Forty eight hours after infection, cells were fixed and analyzed by immunofluorescence imaging as described previously (Riva, L. et al. Discovery of SARS-COV-2 antiviral drugs through large-scale compound repurposing. Nature volume 586, pages113-119, 2020). SARS-COV-2 Nucleocapsid protein (NP) was used to quantify number of infected (NP-positive) cells and DAPI to quantify total number of cells. For each condition, the percentage of infection was calculated as the ratio of the number of infected cells stained for coronavirus NP to number of cells stained with DAPI. Cell viability was measured with MTT assay and viral infection was measured when cells were pre-incubated with the compounds for 2 hours prior to infection and infection was over 48 hours.

### Anti-Cancer Activity of Compounds of the Invention

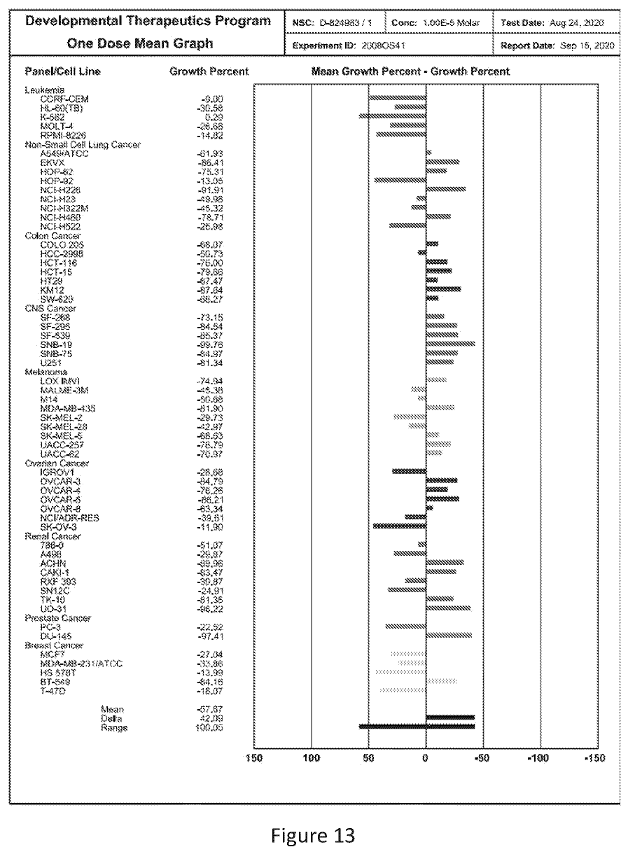

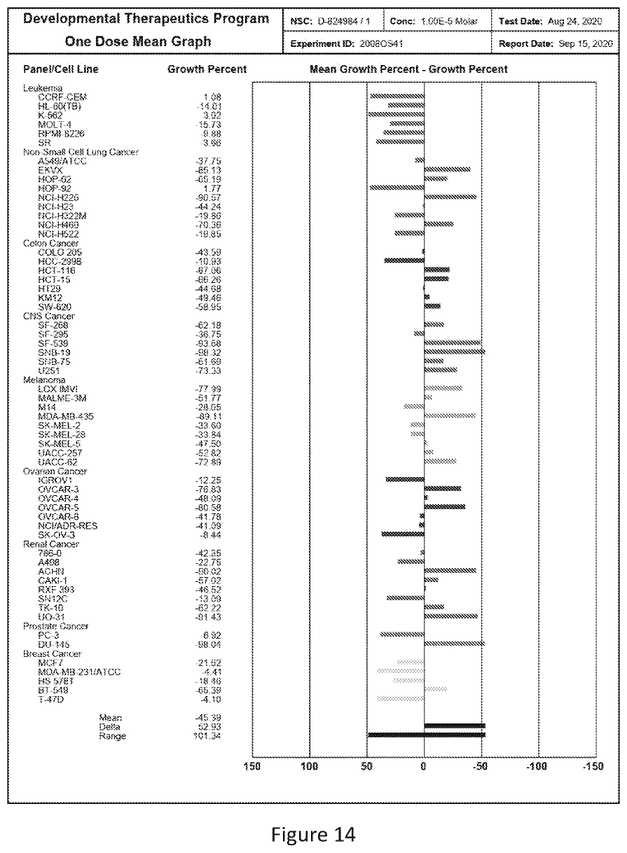

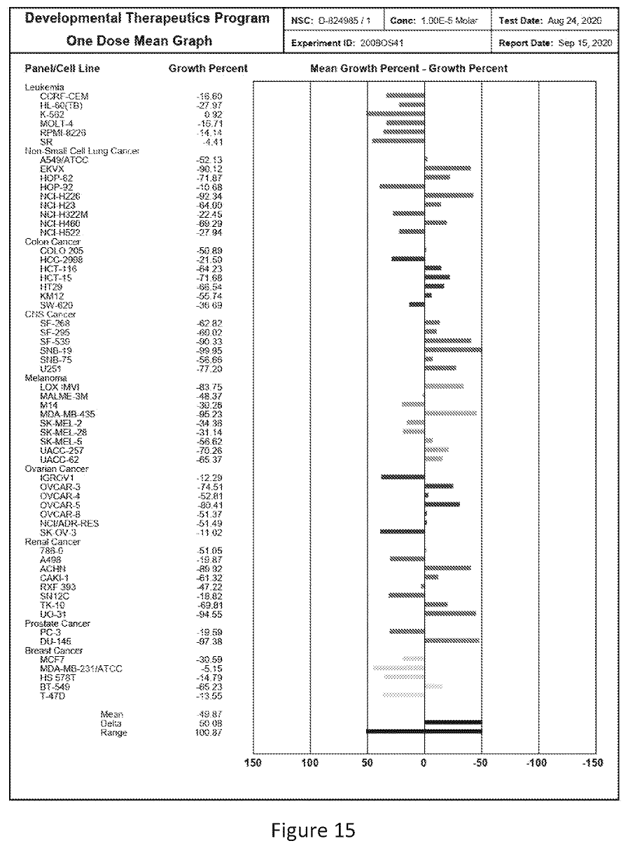

The effects of compound 1, compound 2 and bufalin on the growth of different cancer cells are depicted in FIGS. 13-15. One dose effect of Compounds 1 and 2 of the present invention and bufalin on cancer cell viability, as determined at the NCI, NIH, is shown in FIGS. 13-15 provide evidence for the anti-cancer effects of the synthetic compound. The most sensitive line to the compounds 1 and 2 seem to be SNB-19 and the least sensitive was K562 (see materials and methods for data interpretation).

### Experimental:

In the anti-cancer studies were performed at the NCI, NIH in the one-dose testing of the NCI-60 project (https://dtp.cancer.gov/discovery_development/nci-60/methodology.htm). The numbers reported are growth relative to the no-drug control, and relative to the time zero number of cells. This allows detection of both growth inhibition (values between 0 and 100) and lethality (values less than 0). For example, a value of 100 means no growth inhibition. A value of 40 would mean 60% growth inhibition. A value of 0 means no net growth over the course of the experiment. A value of −40 would mean 40% lethality. A value of −100 means all cells are dead. Hence, the most sensitive line to the two compounds seem to be SNB-19 and the least sensitive was K562.

1-54. (canceled)

55. A compound of general formula (I): 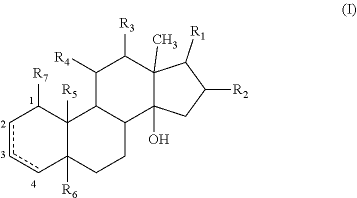
wherein
R₁ is a lactone,
each of R₂, R₃, R₄, R₅, R₆ and R₇, independently of the other, is selected from —H, —OR′, —C₁-C₅alkyl, —C₁-C₅alkylene-hydroxyl and —C(—O)R″,
each of R′ and R″, independently of the other, is selected from —H, —C₁-C₅alkyl, —C₁-C₅alkylene-hydroxyl and —C(═O)C₁-C₅alkyl; and
a bond designated [symbol] is a double bond or a single bond, such that one of the bonds designated [symbol] is a double bond and the other is a single bond.

56. The compound according to claim 55, wherein the lactone is lactone (i) or lactone (ii): 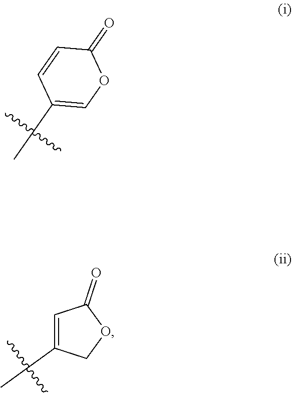 [SMILES: *c1ccc(=O)oc1 ; *C1=CC(=O)OC1]
wherein [symbol] designates a bond of connectivity.

57. The compound according to claim 56, being a compound of the general formulae (Ia) or (Ib) 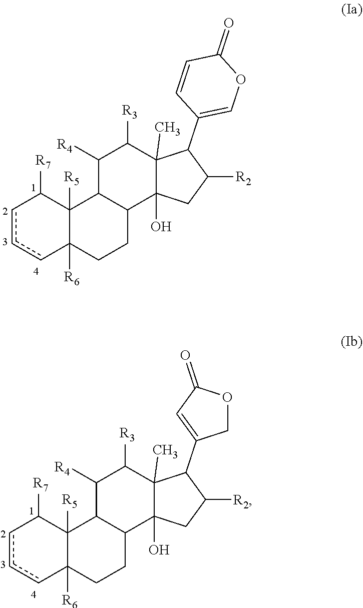
wherein each of each of R₂, R₃, R₄, R₅, R₆, R₇, R′, R″ and [symbol] is as defined in claim 1.

58. The compound according to claim 57, wherein in each of compound of general formula (Ia) and (Ib), R₂ is selected from —H and —OR′, wherein R′ is as defined in claim 55.

59. The compound according to claim 60, wherein R′ is —H or is —C(═O) C₁-C₅alkyl.

60. The compound according to claim 59, wherein the —C(═O)C₁-C₅alkyl is —C(—O)CH₃.

61. The compound according to claim 57, wherein in each compound of general formula (Ia) and (Ib), R₃ is selected from —H and —OH.

62. The compound according to claim 57, wherein in each compound of general formula (Ia) and (Ib), R₄ is selected from —H and —OH.

63. The compound according to claim 57, wherein R₅ is —C₁-C₅alkyl, being optionally a methyl group.

64. The compound according to claim 57, wherein in each compound of general formula (Ia) and (Ib), R₆ is selected from —H and —OH.

65. The compound according to claim 57, wherein in each compound of general formula (Ia) and (Ib), R₇ is selected from —H and —OR′, wherein R′ is selected from —H and —C₁-C₅alkyl.

66. The compound according to claim 57, wherein in each compound of general formula (Ia) and (Ib), the bond between C3 and C2 is a double bond.

67. The compound according to claim 57, wherein in each compound of general formula (Ia) and (Ib), the bond between C3 and C4 is a double bond.

68. A compound having structure (IIa) or (IIb):

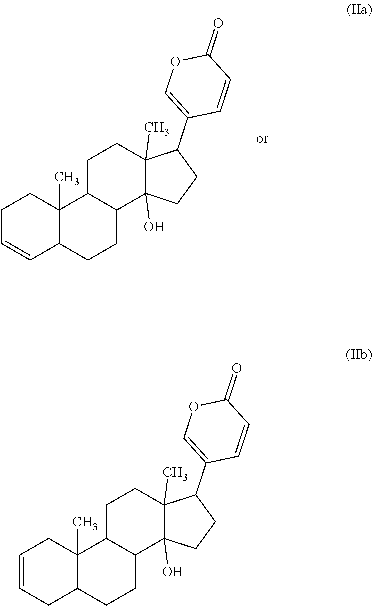 [SMILES: CC12CCC=CC1CCC1C2CCC2(C)C(c3ccc(=O)oc3)CCC12O ; CC12CC=CCC1CCC1C2CCC2(C)C(c3ccc(=O)oc3)CCC12O]

69. A pharmaceutical composition comprising a compound according to claim 55.

70. A method of treatment or prophylaxis of a disease or disorder associated with Na⁺/K⁺ ATPase activity, the method comprising administering to a subject (human or non-human) in need thereof an effective amount of a compound according to claim 55.

71. The method according to claim 70, wherein the disease or disorder is selected from heart failure, atrial fibrillation, fetal tachycardia, supraventricular tachycardia, cor pulmonale, pulmonary hypertension, cancers, neurological diseases and psychiatric diseases.

72. A method of treatment or prophylaxis of cancer, the method comprising administering to a subject (human or non-human) in need thereof an effective amount of a compound according to claim 55.

73. A method of treatment or prophylaxis of a disease or disorder associated with a viral infection, the method comprising administering to a subject (human or non-human) in need thereof an effective amount of a compound according to claim 55.

74. A de-hydroxy cardiac steroid.
